(19) United States
(12) Patent Application Publication
(10) Pub. No.: US 2002/0172624 A1
(43) Pub. Date: Nov. 21, 2002

(54) Scenting device

(76) Inventor: Gernot Busch, Frankfurt (DE)

Correspondence Address: MILDE, HOFFBERG & MACKLIN, LLP, SUITE 460, 10 BANK STREET, WHITE PLAINS, NY, 10606, US

(21) Appl. No.: 10/078,191

(22) Filed: Feb. 19, 2002

(30) Foreign Application Priority Data
May 17, 2001 (DE) 101 24 157.7

(51) Int. Cl. A61L 9/00; A61L 9/02; A61L 9/03

(52) U.S. Cl. 422/124; 422/123

(57) ABSTRACT

A scenting device to simulate and test a pre-determined room climate is presented in which, based on the invention, at least one receptacle is provided in which at least one scent material receptor is positioned that contains a scent material that simulates each room climate to be tested. The receptacle includes at least one aperture from which the scent material diffuses with the help of a gas distribution device, e.g., a small fan. The scent material may then be tested by a tester.

The invention relates to a scenting device to simulate and test a pre-determined room climate.

Scenting devices are known with which rooms may be scented in order to test a pre-determined room climate. For this, a preselected scent is distributed throughout a room. Subsequently, a tester, for example, may enter this room and test the simulated room climate.

With the known scenting device, there is the disadvantage that a room must be completely scented and subsequently neutralized in order to be simulated and tested. This is cost-intensive on the one hand, and time-consuming on the other. Moreover, the tester is also completely scented by this process, i.e., his clothing, hair, and skin, etc. receive the scent. In order to conduct another test of another room climate, the tester must first neutralize his clothing, hair, and skin, etc. Otherwise, no objective test of a new room climate can result. Thus, further testing of a room climate is made considerably more difficult using the known scenting device.

It is therefore the objective of the invention to improve a scenting device in such a manner that the disadvantages known in the state of the art are avoided.

Based on the invention, this task is solved by a scenting device according to the properties of Patent claim 1.

Accordingly, a receptacle is scented using a pre-determined scent material that is present, for example, in a scent material receptor, whereby the issuing scent material is distributed within the receptacle by a gas distribution device such as a small fan, and issues from at least one aperture of the receptacle. Thus, a tester may, for example, test the scent material, and thereby the simulated room climate, directly at an aperture of the receptacle in the most simple manner without scenting the entire room and, for example, without contaminating the clothing or similar of the tester with the scent during the test.

The receptacle is preferably a glass container; however, it is conceivable that the receptacle be made of another suitable material such as plastic or similar material, for example.

A further expansion of the invention may provide for the connection of a funnel-shaped tube to the aperture of the receptacle that conically expands from the aperture in the container so that a tester may simply test a room climate simulated within a receptacle.

It is particularly advantageous if the end of the tube pointed toward the tester includes a nosepiece fitted to his face so that the tester may press his nose against the nosepiece so that the scent material issuing from it may essentially make its way into his nose. Of course, the tube may be shaped in many other ways.

The tube is preferably coated with Teflon or another coating material, for example, for hygienic reasons.

The scent material receptor may be a container, such as a scent can or similar device, for example, in which an absorber soaked with scent material, for example, is provided. The scent material may diffuse from the container into the interior of the receptacle.

Of course, the scent material to be tested may also be passed into the receptacle without a container.

Based on an expansion of the invention, the scent material receptor may include a heating device whereby the diffusion of the scent material is further optimized. It is particularly advantageous if the heating device is connected with a time switch. In this manner, the receptacle may be optimally scented before testing, for example.

The scent material may be optimally distributed or transported in the receptacle by means of the gas distribution device provided in the receptacle. The gas distribution device may include, for example, a fan or similar device. The fan may preferably be positioned within the funnel-shaped tube in order to transport the scent material from the receptacle to the tester. Of course, the gas distribution device or the fan may be provided at another location such as in the interior of the receptacle, for example.

Further advantages and advantageous embodiments of the invention result from the sub-claims and from the following description of the embodiment example, based on illustrations which show:

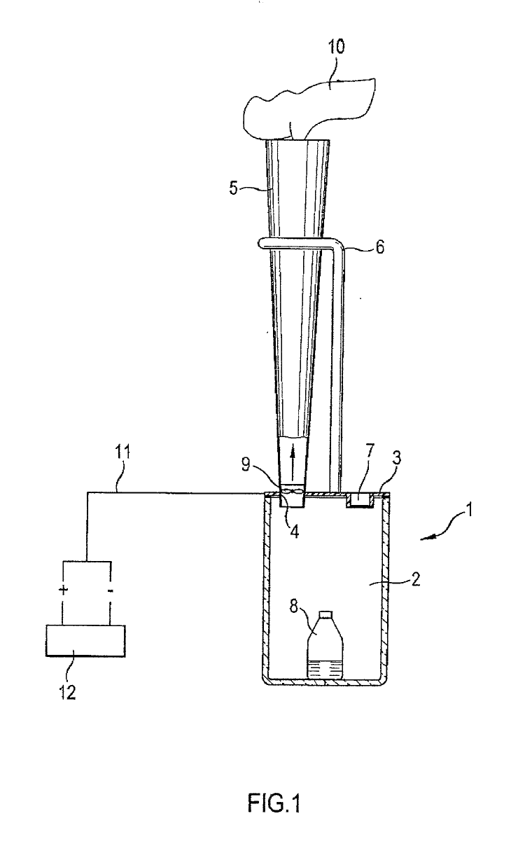

FIG. 1 a schematic view of a scenting device based on the invention;

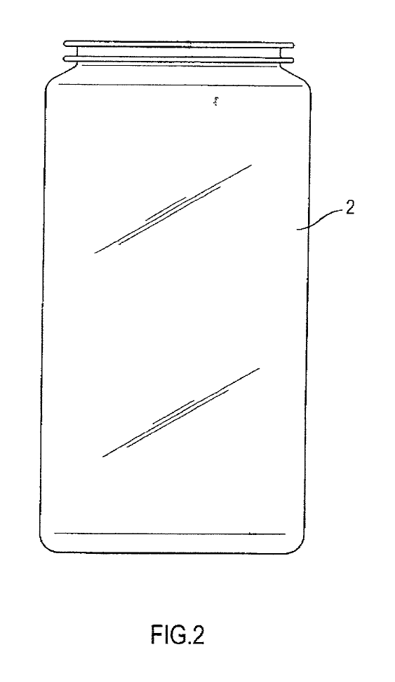

FIG. 2 a side view of a receptacle of the scenting device; and

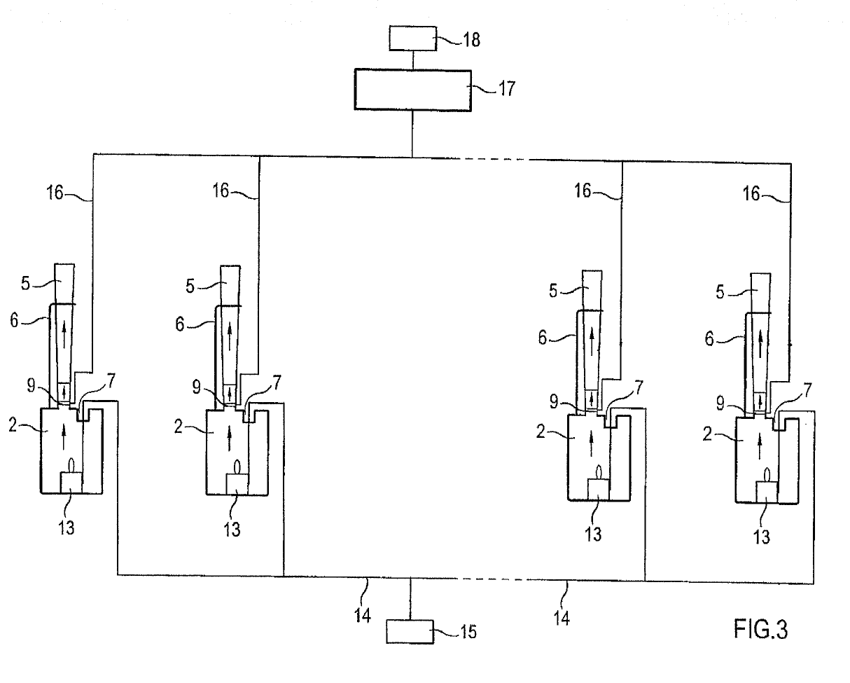

FIG. 3 a schematic view of numerous scenting devices that form a testing device.

FIG. 1 shows a scenting device 1 based on the invention used to simulate and test a pre-determined room climate. The scenting device 1 includes a receptacle that is a glass container 2 in this embodiment example of the invention.

The glass container 2 includes a plastic lid 3 with which it may be closed. The plastic lid 3 has an aperture 4 into which a funnel-shaped tube 5 is inserted. The funnel-shaped tube 5 is secured to the glass container 2 or the plastic lid 3 by means of a holder 6. The plastic lid 3 includes another passage 7 for cables or wires whereby the passage 7 in FIG. 1 is sealed.

A scent material receptor is provided within the glass container 2. In FIG. 1, the scent material receptor includes a bottle 8 filled with scent material. From the bottle 8, the scent material can then diffuse into the interior of the glass container 2 and then simulate a pre-determined room climate in the glass container 2.

A gas distribution device is provided within the funnel-shaped tube 5 that includes a fan 9. The fan 9 is positioned in the area of a first end of the funnel-shaped tube 5 pointed toward the aperture 4. The fan 9 transports the scent material to be tested to a second end of the funnel-shaped tube, at which a tester 10 may test the room climate simulated within the glass container 2. The flow direction of the scent material is shown by an arrow in FIG. 1. The fan 9 is secured within the tube 5 by means of fasteners (not shown), and is connected via an electrical connector line 11 with a low-voltage source 12 used as a power supply.

A nosepiece (not shown) is included at the second end of the funnel-shaped tube 5 so that the diffusing scent material may make its way directly into the nose of the tester 10. Thus, the scenting device based on the invention ensures ideal testing conditions.

FIG. 2 shows a side view of the glass container 2. In this embodiment, it is made of conventional recycling glass.

FIG. 3 shows a testing system for which four scenting devices 1 are provided, by means of which four different room climates might be tested, for example. In this embodiment of the invention, scent material receptors are provided in the glass container 2, each of which includes a heating device 13 in order to enable an optimum diffusion of the scent material. The heating devices 13 are each connected with a common power source 15 via an electrical connector line 14. The connector line is passed through a passage 7 provided in the lid and into the interior of each glass container 2 to the heating devices 13.

The fans 9 of the gas distribution device are connected to a transformer 17 via electrical connector lines 16. The transformer 17 is in turn connected to a power source 18. The flow direction of the scent material is shown by arrows. Identical components are designated using reference numerals consistent with FIG. 1.

This testing system with several scenting devices is used as follows:

The test equipment may, for example, include eight glass containers 2, eight funnel-shaped tubes 5, four scent material receptors with connectors and cables for each heating device 13, five fans 9, a transformer 17 with five connections, four extension lines, and a cleaner for the funnel-shaped tube 5. All parts of the testing system are suitable for continuous operation.

When using a scent material receptor it should be pre-heated using a heating device 13 for about four hours before the beginning of testing. A time switch is preferably used for this.

A scent receptor is positioned within each glass container 2 whereby the electrical connector lines 14 may be passed through a corresponding passage 7 in the plastic lid 3 of the glass container 2 in order to connect them with a power source 15.

Subsequently, the fan 9 of the gas distribution device can be secured to the aperture 4 of each glass container 2. Each fan 9 is connected with a transformer 17 via an electrical connector line 16 that in turn is connected to a power source 18. Then, a funnel-shaped tube 5 is inserted into the aperture 4 of each glass container 2 above each fan 9, whereby the tube 5 is secured to the holder 6 on the glass container 2 or the plastic lid 3.

The corresponding room climate at the free ends of the funnel-shaped tubes 5 can be tested by a tester by means of the diffusion of each scent material into each glass container 2.

1. Scenting device to simulate and test a pre-determined room climate, characterized in that at least one receptacle is provided, with at least one gas distribution device and at least one scent material receptor, and that at least one aperture (4) is provided for the testing scent material.

2. Scenting device as in claim 1, characterized in that the receptacle is a glass container (2) whereby the scent material receptor is provided within the glass container (2) to diffuse a scent material to be tested, and that the gas distribution device is so positioned in the area of the aperture (4) that the scent material to be tested may be transported to the tester (10).

3. Scenting device as in one of claims 1 or 2, characterized in that the aperture (4) is provided in the receptacle for the diffusion of the scent material, whereby the aperture (4) is connected with a funnel-shaped tube (5) in which the gas distribution device is positioned.

4. Scenting device as in claim 3, characterized in that an end of the funnel-shaped tube (5) pointed away from the aperture (4) includes a nosepiece that is so shaped that the diffusing scent material may make its way directly into the nose of the tester (10).

5. Scenting device as in one of claims 3 or 4, characterized in that the funnel-shaped tube (5) is coated with Teflon.

6. Scenting device as in one of claims 1 through 5, characterized in that the scent material receptor is a container (8) with a pre-determined scent material from which scent material diffuses.

7. Scenting device as in claim 6, characterized in that the scent material receptor includes a heating device (13).

8. Scenting device as in claim 7, characterized in that the heating device (13) may be operated using a time switch.

9. Scenting device as in one of claims 7 or 8, characterized in that the heating device (13) is connected to a power source (15) via an electrical connector line (14).

10. Scenting device as in one of claims 1 through 9, characterized in that the gas distribution device includes a fan (9).

11. Scenting device as in claim 10, characterized in that the fan (9) is connected to a low-voltage power source (12) via an electrical connector line (11).

12. Scenting device as in one of claims 10 or 11, characterized in that the fan (9) is connected to a power source (18) via an electrical connector line (16) and via a transformer (17).
